(19) United States
(12) Patent Application Publication
(10) Pub. No.: US 2017/0362515 A1
(43) Pub. Date: Dec. 21, 2017

(54) PROCESS FOR THE PRODUCTION OF AROMATICS FROM BIOMASS

(71) Applicant: SABIC GLOBAL TECHNOLOGIES B.V., Bergen op Zoom (NL)

(72) Inventors: Flaiyh Al-Anazi, Riyadh (SA); Syed Hashmi, Riyadh (SA)

(21) Appl. No.: 15/532,733

(22) PCT Filed: Dec. 2, 2015
(86) PCT No.: PCT/IB2015/059295

§ 371 Date: Jun. 2, 2017

Related U.S. Application Data
(60) Provisional application No. 62/087,496, filed on Dec. 4, 2014.

(51) Int. Cl. C10G 50/00; C10B 53/02; C12P 5/02

(52) U.S. Cl. CPC C10G 50/00; C12P 5/023; C10B 53/02; C10G 2300/1014; C10G 2400/30; C10G 2300/1018

(57) ABSTRACT

A method of producing an aromatic chemical, comprises: providing a feedstock comprising biomass to a first reactor to produce a first product stream, wherein the first product stream comprises methane and carbon dioxide; combining the first product stream with a recycle stream to form a second reactor feed stream; passing the second reactor feed stream through a second reactor to produce a second product stream comprising aromatics and hydrogen gas; recovering aromatics from the second product stream to create a recovery stream depleted of aromatics; combining the recovery stream with a stream comprising carbon dioxide to form a combined recovery stream; passing the combined recovery stream to a third reactor to produce the recycle stream comprising gas; and forming an aromatic chemical from the second product stream.

### BACKGROUND

Aromatic chemicals (aromatics), such as benzene, have many uses in the chemical industry and demand for these compounds continue to grow each year. In the production of aromatics, a petroleum feed source can be subjected to a variety of manufacturing processes including catalytic reforming, toluene hydrodealkylation, toluene disproportionation, and steam cracking. Alternatively, dehydrocyclization processes can convert methane (CH₄) to aromatics.

### SUMMARY

Disclosed in various embodiments, are processes for the purification of terephthalic acid.

A method of producing an aromatic chemical, comprises: providing a feedstock comprising biomass to a first reactor to produce a first product stream, wherein the first product stream comprises methane and carbon dioxide; combining the first product stream with a recycle stream to form a second reactor feed stream; passing the second reactor feed stream through a second reactor to produce a second product stream comprising aromatics and hydrogen gas; recovering aromatics from the second product stream to create a recovery stream depleted of aromatics; combining the recovery stream with a stream comprising carbon dioxide to form a combined recovery stream; passing the combined recovery stream to a third reactor to produce the recycle stream comprising gas; and forming an aromatic chemical from the second product stream.

A method of producing an aromatic chemical, comprises: providing a feedstock comprising biomass to a first reactor to produce a first product stream, wherein the first product stream comprises methane and carbon dioxide; combining the first product stream with a recycle stream to form a second reactor feed stream; passing the second reactor feed stream through a dehydroaromatization reactor to produce a second product stream comprising aromatics and hydrogen gas; recovering aromatics from the second product stream to create a recovery stream depleted of aromatics; combining the recovery stream with a stream comprising carbon dioxide to form a combined recovery stream; passing the combined recovery stream to a third reactor to produce a third product stream comprising water and gas; forming an aromatic chemical from the second product stream; and recovering methane from the third product stream to form the recycle stream.

A method of producing an aromatic chemical, comprises: supplying a feedstock comprising biomass to a digester, wherein digestion occurs at 20° C. to 50° C. to form a first product stream; passing the first reactor outlet stream to a first separator, wherein the first reactor outlet stream comprises 55 wt. % to 70 wt. % methane and 30 wt. % to 45 wt. % carbon dioxide and wherein the first separator separates the first reactor outlet stream into a first product stream comprising methane and a diverted stream comprising carbon dioxide; recovering the first product stream from the first separator; combining the first product stream with a recycle stream to form a second reactor feed stream; passing the second reactor feed stream through a second reactor to convert the methane to aromatics and hydrogen through a dehydrocyclization reaction and to hydrocarbons with a dehydrogenation-coupling reaction in the second reactor to form a second product stream; feeding the second product stream to a condenser to separate the aromatics from the second product stream to form an aromatic stream and an aromatic depleted product stream; combining the aromatic depleted product stream with hydrogen to form a combined recovery stream; sending the combined recovery stream to a methanation reactor to form a third product stream; feeding the third product stream to a second separator; and separating the third product stream to form a stream comprising water and the recycle stream comprising methane in the second separator.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 is an illustration of an embodiment of a process for the production of aromatics from a biomass feedstock.

FIG. 2 is an illustration of an embodiment of a process for the production of aromatics from a biomass feedstock where the recovery stream is combined with a diverted stream and recycled.

FIG. 3 is an illustration of an embodiment of a process for the production of aromatics from a biomass feedstock where the recovery stream is further separated prior to combining with a diverted stream and recycled.

### DETAILED DESCRIPTION

As the cost of petroleum feed sources increases, the economic viability of the production of aromatics from petroleum diminishes. Due at least in part to economic factors, it is believed that methane feed sources can displace petroleum and can become an important source in the production of aromatic compounds.

Direct conversion of methane (CH₄) to aromatics by dehydrocyclization processes can produce aromatics with hydrogen as a by-product. However, thermodynamic limitations of dehydrocyclization reactions can reduce the yield of aromatics as unconverted methane in the product stream can limit conversion. Furthermore, separation of hydrogen from the aromatic product can entail an expensive separation process (e.g., cryogenic separation, pressure-swing absorption, or a combination of both) which can reduce the economic benefit of processes utilizing a non-petroleum feedstock.

Thus, there is a desire in the chemical industry to produce aromatic chemicals, such as benzene, toluene, naphthalene, and/or other aromatics, from renewable feedstocks (e.g., biomass) in a cost competitive manner. Accordingly, a need exists for a process that does not require hydrogen separation from the aromatic product stream, and which does not compromise the thermodynamics of methane dehydrocyclization.

Disclosed herein is a process for the production of an aromatic chemical from a biomass feedstock. The process can include introducing a biomass feedstock into a first reactor. The biomass feedstock can include any biologically-produced matter comprising carbon and hydrogen. The biomass feedstock can include any biologically-produced matter that is capable of conversion to methane. For example, the biomass feedstock can include material derived from vegetation, aquatic sources (e.g., aquaculture), forestry, agriculture, animal waste, or a combination including at least one of the foregoing. The biomass can be in a liquid, solid, or gaseous state when it can be introduced to the first reactor in any suitable fashion (e.g., loaded, poured, flowed, conveyed, or the like). The first reactor can include any reactor capable of recovering methane from the biomass feedstock. The first reactor can include multiple stages for optimizing production of methane from the biomass feedstock. The first reactor can include a stirred reactor, a plug flow reactor, or a batch reactor. The first reactor can include bacteria for converting the biomass feedstock to methane, e.g., methanogenic bacteria. The first reactor can include an anaerobic digester, such as a plug flow digester, a complete mix digester, and the like.

The biomass feedstock can be reacted in the first reactor under conditions effective to produce a first product stream including methane and carbon dioxide. The first reactor can be operated at 10° C. to 60° C., for example, 20° C. to 50° C. The residence time of the biomass feedstock in the first reactor can be 1 to 20 days, for example, 1 to 15 days. The first product stream can be in gas phase. The first product stream can include 50 to 95 weight percent (wt. %) methane, for example, 55 wt. % to 90 wt. %, or 55 wt. % to 70 wt. %. The first product stream can include 5 wt. % to 50 wt. % carbon dioxide, for example, 10 wt. % to 45 wt. %, or 30 wt. % to 45 wt. %.

The process can include separating the first reactor outlet stream in a first separator to form the first product stream including methane and a diverted stream including carbon dioxide. The diverted stream can be diverted from the first product stream. The first separator can include any separating apparatus capable of separating the first reactor outlet stream into the first product stream including methane and the diverted stream including carbon dioxide. Such separation apparatus can include a cryogenic condenser, pressure swing absorber, temperature swing absorber, gas/liquid contactor, scrubber, and the like. The process can include a first separator such that the methane concentration of the second reactor feed stream can be increased by separating and diverting carbon dioxide from the first reactor outlet stream. In an embodiment, the diverted stream, separated from the first reactor outlet stream, can be sent to another section of the process (e.g., it can be sent to another reactor for further processing, such as a methanation reactor).

The process can include combining the first product stream with a recycle stream to form a second reactor feed stream. The recycle stream can include methane.

Combining as used herein includes bringing together two or more streams. Two or more streams can be combined outside or inside the boundary of a unit (e.g., a reactor, separator, recovery device, and the like). For example, combining includes joining two or more conduits, each conveying a process stream, into a single conduit (e.g., manifold, reactor, pipe, vessel, and the like). Combining includes, but does not require, mixing, as in static or dynamic mixing of the combining streams.

The process can include passing the second reactor feed stream through a second reactor to produce a second product stream including aromatics and hydrogen gas. The process can include contacting the second reactor feed stream with a catalyst under conditions effective to form the second product stream. The second reactor can include a packed bed reactor. The second reactor can include a packing material. The packing material can provide a catalyst support structure where a catalyst can be immobilized on the packing material. The catalyst can include a bi-functional catalyst including a metal catalyst disposed on a zeolite support structure. The metal can include molybdenum, tungsten, ruthenium, iron, cobalt, nickel, copper, silver, zinc, chromium, tin, or a combination including at least one of the foregoing. The zeolite support can include a pentasil type zeolite family, modified pentasil type zeolite family, other medium pore zeolites (e.g., zeolite beta and zeolite MCM-22), or a combination including at least one of the foregoing.

Chemical reactions in the second reactor can include dehydrogenation, cyclization, dehydrogenation-coupling, or a combination including at least one of the foregoing. Reaction in the second reactor can include simultaneous dehydrogenation and cyclization (e.g., a dehydrocyclization process) to form aromatics and hydrogen. Reaction in the second reactor can include dehydrogenation-coupling to form hydrocarbons (e.g., non-aromatic hydrocarbons). The aforementioned catalyst, and following second reactor conditions, can promote the desired reaction processes. The second reactor can be operated at a temperature of 400° C. to 1000° C., for example, 500° C. to 850° C., or 700° C. to 750° C. The second reactor can be operated at an absolute (abs) pressure of 0.2 to 5 atmospheres (atm (abs)) (20 to 507 kilopascal kPa (abs)), for example, 0.5 to 2 atm (abs) (50.7 to 203 kPa (abs)). The second reactor can be operated with a gas hourly space velocity (GHSV) of the second reactor feed stream from 400 to 8,000 GHSV, for example, 500 to 7,000 GHSV. Gas hourly space velocity (GHSV) as used herein can refer to the volume of gas fed to the reactor per volume of catalyst, including catalyst support material, in the reactor per hour. The GHSV can be calculated by dividing the volumetric flow rate of gas fed to the reactor by the combined volume of the catalyst and catalyst support material contained within the reactor.

The process can include recovering aromatics from the second product stream to create a recovery stream depleted of aromatics. An aromatics recovery unit can be employed to separate the second product stream into the recovery stream (depleted of aromatics) and an aromatic product stream. The aromatics recovery unit can be a chemical separation unit, such as a condenser, cryogenic separator, gas/liquid contactors, or the like. The recovery stream can include methane (e.g., methane not reacted in the second reactor) and hydrogen (e.g., by-product hydrogen from the dehydrocyclization process). The hydrogen content of the recovery stream can be further increased by combining a first supplemental hydrogen stream including hydrogen with the recovery stream. Supplemental hydrogen can be supplied from any suitable process. For example, supplemental hydrogen can be derived from a renewable source (e.g. water splitting processes such as electrolysis or biological material), from processes including hydrocarbon reforming (e.g. methane reforming), cracking (e.g. hydrocarbon cracking), dehydrogenation processes, hydrogen liberating chemical processes, or a combination including at least one of the foregoing.

The process can include separating the recovery stream in a third separator to form a methane recovery stream including methane and a hydrogen recovery stream including hydrogen. The hydrogen recovery stream can be combined with the first supplemental hydrogen stream, the stream containing carbon dioxide (e.g. the diverted stream), or a combination including at least one of the foregoing. The methane recovery stream can be combined with the first product stream, the third product stream, a recycle stream including methane (derived from the third product stream as described in the following), the second supplemental hydrogen stream, or a combination including at least one of the foregoing.

The process can include combining the recovery stream (aromatic depleted stream with or without supplemental hydrogen) with a stream including carbon dioxide to form a combined recovery stream. The stream including carbon dioxide can be derived from the first product stream. For example, the stream including carbon dioxide can include the diverted stream, separated from the first reactor outlet stream in the first separator as previously described. Alternatively, the stream including carbon dioxide can include a fresh carbon dioxide supply, a stream from another process (e.g., separation process, reaction process, including combustion, reforming, water gas shift processes, or the like), or a combination including at least one of the foregoing.

The process can include passing the combined recovery stream to a third reactor to produce a third product stream comprising water and gas. The process can include contacting the combined recovery stream with a catalyst under conditions effective to form the third product stream including water and gas. The third product stream can include methane gas and gas phase water. Methane gas in the third product stream can include unreacted methane from the combined recovery stream, methane synthesized in the third reactor, or a combination including at least one of the foregoing. The third reactor can include a packed bed reactor. The third reactor can include a methanation catalyst disposed on a catalyst support material. Methanation catalysts can include rhodium, palladium, platinum, iridium, ruthenium, cobalt, nickel, iron, or a combination including at least one of the foregoing. The catalyst support material can include an inorganic oxide (e.g., titanium oxide, silicon oxide, aluminum oxide, cesium oxide, zirconium oxide, and the like) onto which the catalyst can be immobilized.

Methanation in the third reactor can be carried out under conditions effective to form the third product stream including methane gas and water. The third reactor can be operated at a temperature of 200° C. to 600° C., for example, 300° C. to 575° C., or, 500° C. to 575° C. The third reactor can be operated at a gauge (g) pressure of 0 to 680 atm (g) (0 to 69 megaPascal (MPa) (g)). The methanation can be conducted with a GHSV of the combined recovery stream of 200 to 10,000 GHSV or about 600 to 5,000 GHSV.

The third product stream can be combined with the first product stream to form the second reactor feed stream. The third product stream can be separated in a second separator to form a water removal stream and a recycle stream including methane. The recycle stream can be combined with a methane recovery stream from the third separator, the first product stream, a second supplemental hydrogen stream, or a combination including at least one of the foregoing, to form the second reactor feed stream. The water removal stream can be a liquid phase stream flowing from the second separator. The recycle stream including methane can be a gas phase stream flowing from the second separator.

The process can include forming an aromatic chemical from the second product stream. The aromatics recovered from the aromatics recovery unit can include benzene, toluene, naphthalene, or a combination including at least one of the foregoing.

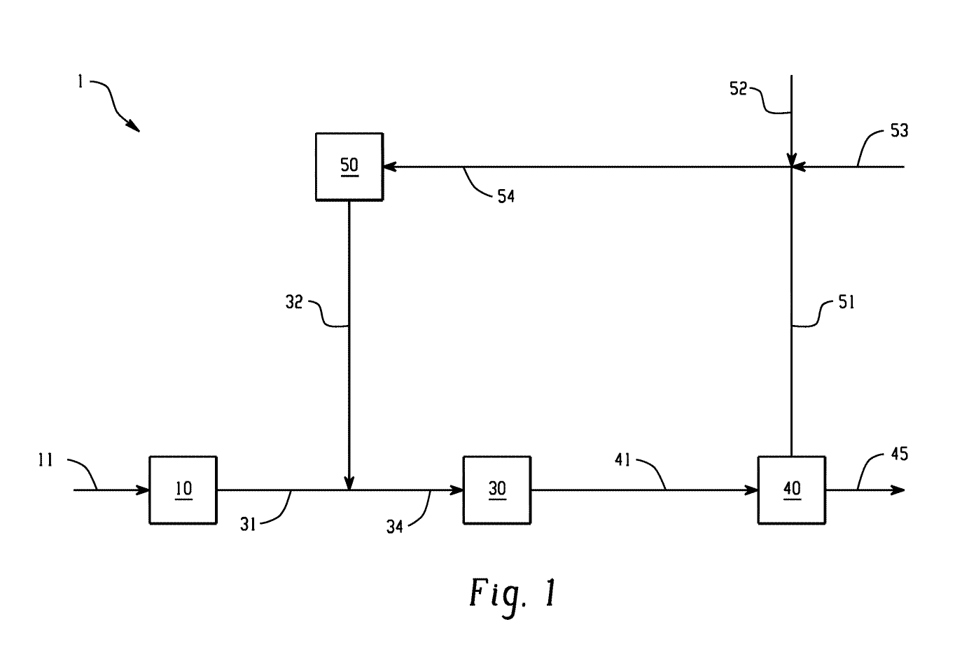

FIG. 1 is an illustration of a process 1 for producing aromatics. The process 1 includes a biomass feedstock 11 which is introduced to a first reactor 10. The biomass feedstock 11 is reacted in the first reactor 10 to form a first product stream 31 which can be combined with a recycle stream 32 to form a second reactor feed stream 34. The second reactor feed stream 34 can be passed through a second reactor 30 where it can undergo dehydrogenation, cyclization, dehydrogenation-coupling, or a combination including at least one of the foregoing to form a second product stream 41. A stream including aromatics 45 can be removed from the second product stream 41 in an aromatic recovery unit 40 and a recovery stream 51, depleted of aromatics, can be returned back to the second reactor 30. The recovery stream 51 can include methane and hydrogen. The recovery stream 51 can be combined with a stream including carbon dioxide 52, to form a combined recovery stream 54. The methane content of the combined recovery stream 54 can be increased by passing it through a third reactor 50 such as a methanation reactor. The recycle stream 32 including methane can be combined with the first product stream 31 to form the second reactor feed stream 34. A first supplemental hydrogen stream 53 can provide additional hydrogen to the combined recovery stream 54 to enhance methanation in the third reactor 50. The stream including aromatics 45 can include an aromatic chemical such as benzene, toluene, xylene, naphthalene, or a combination including at least one of the foregoing.

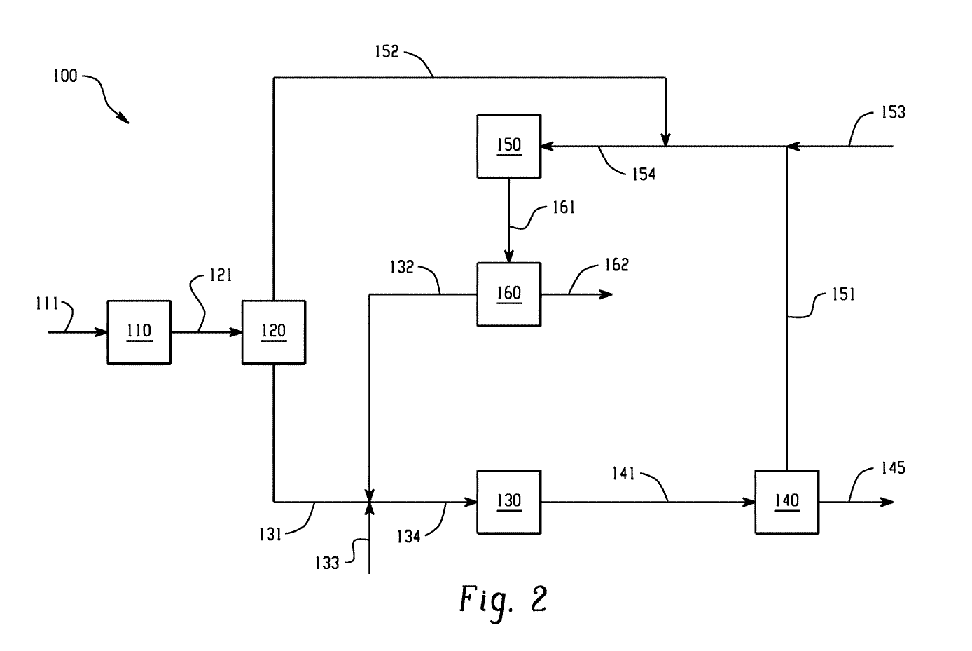

FIG. 2 is an illustration of a process 100 for producing aromatics. The process 100 includes a biomass feedstock 111 which is introduced to a first reactor 110. The biomass feedstock 111 can be reacted in the first reactor 110 to form a first reactor outlet stream 121 which can be separated in a first separator 120, to form a diverted stream 152, including carbon dioxide, and a first product stream 131, including methane. The first product stream 131 can be combined with a recycle stream 132, a second supplemental hydrogen stream 133, or a combination comprising at least one of the foregoing, to form a second reactor feed stream 134. The second reactor feed stream 134 can be passed through a second reactor 130 where it can undergo dehydrogenation, cyclization, dehydrogenation-coupling, or a combination including at least one of the foregoing to form a second product stream 141. A stream including aromatics 145 can be removed from the second product stream 141 in an aromatic recovery unit 140 and a recovery stream 151, depleted of aromatics, can be returned back to the second reactor 130 via recycle stream 132. The recovery stream 151 can include methane and hydrogen. The recovery stream 151 can be combined with the diverted stream 152, including carbon dioxide, a first supplemental hydrogen stream 153, or a combination including at least one of the foregoing, to form a combined recovery stream 154. In an embodiment, the combined recovery stream 154 can be formed within the third reactor 150 (e.g., where the third reactor 150 has inlet ports for conveying the recovery stream 151, the diverted stream 152, the first supplemental hydrogen stream 153, or a combination including at least one of the forgoing). The methane content of the combined recovery stream 154 can be increased by passing it through a third reactor 150 such as a methanation reactor. The methane concentration of the recycle stream 132 can be further increased by removing water from a third product stream 161 in a second separator 160 to form the recycle stream 132 and a water removal stream 162. The recycle stream 132 including methane can be combined with the first product stream 131, a second supplemental hydrogen stream 133, or a combination including at least one of the foregoing, to form the second reactor feed stream 134. A first supplemental hydrogen stream 153 can provide additional hydrogen to the combined recovery stream 154 to enhance methanation in the third reactor 150. The stream including aromatics 145 can include an aromatic chemical such as benzene, toluene, xylene, naphthalene, or a combination including at least one of the foregoing.

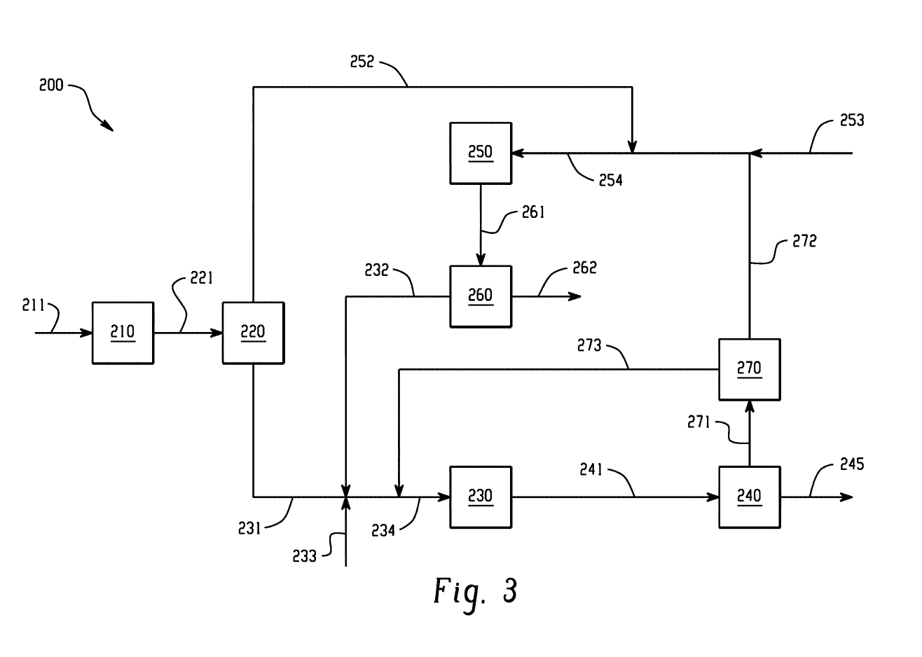

FIG. 3 is an illustration of a process 200 for producing aromatics. The process 200 includes a biomass feedstock 211 which is introduced to a first reactor 210. The biomass feedstock 211 can be reacted in the first reactor 210 to form a first reactor outlet stream 221 which can be separated in a first separator 220, to form a diverted stream 252, including carbon dioxide, and a first product stream 231, including methane. The first product stream 231 can be combined with a recycle stream 232, a second supplemental hydrogen stream 233, a methane recovery stream 273, or a combination including at least one of the foregoing, to form a second reactor feed stream 234. The second reactor feed stream 234 can be passed through a second reactor 230 where it can undergo dehydrogenation, cyclization, dehydrogenation-coupling, or a combination including at least one of the foregoing to form a second product stream 241. A stream including aromatics 245 can be removed from the second product stream 241 in an aromatic recovery unit 240 and a recovery stream 271, depleted of aromatics, can be returned back to the second reactor 230 via recycle stream 232. The recovery stream 271 can include methane and hydrogen. The recovery stream 271 can be separated in a third separator 270 to form a methane recovery stream 273 and a hydrogen recovery stream 272. The hydrogen recovery stream 272 can be combined with the diverted stream 252, including carbon dioxide, a first supplemental hydrogen stream 253, or a combination including at least one of the foregoing, to form a combined recovery stream 254. In an embodiment, the combined recovery stream 254 can be formed within the third reactor 250 (e.g., where the third reactor 250 has inlet ports for conveying the hydrogen recovery stream 272, the diverted stream 252, the first supplemental hydrogen stream 253, or a combination including at least one of the foregoing). The methane content of the combined recovery stream 254 can be increased by passing it through a third reactor 250 such as a methanation reactor. The methane concentration of the recycle stream 232 can be further increased by removing water from a third product stream 261 in a second separator 260 to form the recycle stream 232 and a water removal stream 262. The recycle stream 232 including methane can be combined with the first product stream 231, the methane recovery stream 273, the second supplemental hydrogen stream 233, or a combination including at least one of the foregoing, to form the second reactor feed stream 234. The first supplemental hydrogen stream 253 can provide additional hydrogen to the combined recovery stream 254 to enhance methanation in the third reactor 250. The stream including aromatics 245 can include an aromatic chemical such as benzene, toluene, xylene, naphthalene, or a combination including at least one of the foregoing.

Aromatics synthesized from methane in other processes can require cryogenic separation, pressure swing adsorption (PSA), or a combination of the foregoing processes to separate hydrogen from the aromatic product stream. The present subject matter can overcome this drawback and shift the thermodynamic conditions of the process in favor of higher aromatic conversion. Without wishing to be bound by theory, it is thought that by increasing the concentration of methane in the second reactor (e.g., dehydrocyclization reactor) the conditions favor the formation of aromatics, thus improving the product conversion. Therefore, by subjecting the recovery stream (stream depleted of aromatics) to reaction in a third reactor (e.g., methanation reactor) the methane content of the recycled stream can be increased. Furthermore, combining the diverted stream including carbon dioxide (separated from the first product stream) with the recovered stream in the third reactor the methane content of the recycled stream can be further increased and allows for more efficient use of the feedstock. Moreover, by removing water from the third product stream (e.g., methanation product stream) the methane concentration of the recycle stream can be increased. Thus, it is believed that including a third reactor, capable of converting hydrogen and carbon dioxide to methane in the process, can increase the methane content of the recycle stream which can improve conversion of aromatics in the second reactor.

The processes disclosed herein can include at least the following embodiments:

Embodiment 1: A method of producing an aromatic chemical, comprising: providing a feedstock comprising biomass to a first reactor to produce a first product stream, wherein the first product stream comprises methane and carbon dioxide; combining the first product stream with a recycle stream to form a second reactor feed stream; passing the second reactor feed stream through a second reactor to produce a second product stream comprising aromatics and hydrogen gas; recovering aromatics from the second product stream to create a recovery stream depleted of aromatics; combining the recovery stream with a stream comprising carbon dioxide to form a combined recovery stream; passing the combined recovery stream to a third reactor to produce the recycle stream comprising gas; and forming an aromatic chemical from the second product stream.

Embodiment 2: The method of Embodiment 2, wherein the biomass comprises a material selected from vegetation, an aquatic crop, forestry, agricultural residue, animal waste, or a combination comprising at least one of the foregoing.

Embodiment 3: The method of any of Embodiments 1-2, wherein the aromatic chemical is benzene, toluene, xylene, naphthalene, or a combination comprising at least one of the foregoing.

Embodiment 4: The method of any of Embodiments 1-3, wherein the gas comprises methane.

Embodiment 5: The method of Embodiment 4, wherein the methane gas comprises synthetic methane, unconverted methane, or a combination comprising at least one of the foregoing.

Embodiment 6: The method of any of Embodiments 1-5, wherein the recycle stream further comprises water; and further comprising separating the water from the recycle stream.

Embodiment 7: The method of any of Embodiments 1-6, wherein the second reactor is a dehydroaromatization reactor.

Embodiment 8: The method of any of Embodiments 1-7, wherein the first product stream comprises 55 wt. % to 70 wt. % methane and 40 wt. % to 45 wt. % carbon dioxide.

Embodiment 9: The method of any of Embodiments 1-8, further comprising reacting the second reactor feed stream with a catalyst in the second reactor to form the second product stream.

Embodiment 10: The method of Embodiment 9, wherein the catalyst comprises a metal catalyst.

Embodiment 11: The method of Embodiment 10, wherein the metal is selected from molybdenum, tungsten, ruthenium, iron, cobalt, nickel, copper, silver, zinc, chromium, tin, or a combination comprising at least one of the foregoing.

Embodiment 12: The method of Embodiment 11, wherein the metal catalyst is a zeolite supported metal catalyst.

Embodiment 13: The method of any of Embodiments 1-12, further comprising dehydrogenating the second product stream and/or cyclizating of the second product stream.

Embodiment 14: The method of Embodiment 13, wherein the dehydrogenation and/or cyclization of the second product occurs at a temperature of 400° C. to 1,000° C.

Embodiment 15: The method of Embodiment 14, wherein the dehydrogenation and/or cyclization of the second product occurs at a pressure of 0.02 MegaPascals to 0.5 MegaPascals.

Embodiment 16: The method of Embodiment 15, wherein the dehydrogenation and/or cyclization of the second product occurs at a pressure or gaseous hourly space velocity of the feed gas measured in volumes of gas per volume of catalyst per hour of 400 gaseous hourly space velocity to 8,000 gaseous hourly space velocity.

Embodiment 17: The method of any of Embodiments 1-16, further comprising contacting a methanation catalyst with the combined recovery stream to produce the third product stream.

Embodiment 18: The method of Embodiment 17, wherein the methanation catalyst is selected from ruthenium, cobalt, nickel, iron, or a combination comprising at least one of the foregoing.

Embodiment 19: The method of any of Embodiments 1-18, wherein the third product stream is formed at a temperature of 200° C. to 600° C.

Embodiment 20: The method of any of Embodiments 1-19, wherein the third product stream is formed at a pressure of 0 MegaPascals to 75 MegaPascals.

Embodiment 21: A method of producing an aromatic chemical, comprising: providing a feedstock comprising biomass to a first reactor to produce a first product stream, wherein the first product stream comprises methane and carbon dioxide; combining the first product stream with a recycle stream to form a second reactor feed stream; passing the second reactor feed stream through a dehydroaromatization reactor to produce a second product stream comprising aromatics and hydrogen gas; recovering aromatics from the second product stream to create a recovery stream depleted of aromatics; combining the recovery stream with a stream comprising carbon dioxide to form a combined recovery stream; passing the combined recovery stream to a third reactor to produce a third product stream comprising water and gas; forming an aromatic chemical from the second product stream; and recovering methane from the third product stream to form the recycle stream.

Embodiment 22: A method of producing an aromatic chemical, comprising: supplying a feedstock comprising biomass to a digester, wherein digestion occurs at 20° C. to 50° C. to form a first product stream; passing the first reactor outlet stream to a first separator, wherein the first reactor outlet stream comprises 55 wt. % to 70 wt. % methane and 30 wt. % to 45 wt. % carbon dioxide and wherein the first separator separates the first reactor outlet stream into a first product stream comprising methane and a diverted stream comprising carbon dioxide; recovering the first product stream from the first separator; combining the first product stream with a recycle stream to form a second reactor feed stream; passing the second reactor feed stream through a second reactor to convert the methane to aromatics and hydrogen through a dehydrocyclization reaction and to hydrocarbons with a dehydrogenation-coupling reaction in the second reactor to form a second product stream; feeding the second product stream to a condenser to separate the aromatics from the second product stream to form an aromatic stream and an aromatic depleted product stream; combining the aromatic depleted product stream with hydrogen to form a combined recovery stream; sending the combined recovery stream to a methanation reactor to form a third product stream; feeding the third product stream to a second separator; and separating the third product stream to form a stream comprising water and the recycle stream comprising methane in the second separator.

Embodiment 23: The method of Embodiment 22, further comprising contacting the second reactor feed stream with a catalyst in the second reactor.

Embodiment 24: The method of Embodiment 23, wherein the catalyst is a zeolite functional metal catalyst.

Embodiment 25: The method of any of Embodiments 22-24, further comprising adding the reaction products of the first product stream to the methanation reactor to produce the third product stream.

Embodiment 26: The method of any of Embodiments 22-25, further comprising contacting the combined recovery stream with a catalyst selected from ruthenium, cobalt, nickel, iron, or a combination comprising at least one of the foregoing to produce the third product stream.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to distinguish one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

As used herein, the term “hydrocarbyl” and “hydrocarbon” refers broadly to a substituent comprising carbon and hydrogen, optionally with 1 to 3 heteroatoms, for example, oxygen, nitrogen, halogen, silicon, sulfur, or a combination thereof; “alkyl” refers to a straight or branched chain, saturated monovalent hydrocarbon group; “alkylene” refers to a straight or branched chain, saturated, divalent hydrocarbon group; “alkylidene” refers to a straight or branched chain, saturated divalent hydrocarbon group, with both valences on a single common carbon atom; “alkenyl” refers to a straight or branched chain monovalent hydrocarbon group having at least two carbons joined by a carbon-carbon double bond; “cycloalkyl” refers to a non-aromatic monovalent monocyclic or multicylic hydrocarbon group having at least three carbon atoms, “cycloalkenyl” refers to a non-aromatic cyclic divalent hydrocarbon group having at least three carbon atoms, with at least one degree of unsaturation; “aryl” refers to an aromatic monovalent group containing only carbon in the aromatic ring or rings; “arylene” refers to an aromatic divalent group containing only carbon in the aromatic ring or rings; “alkylaryl” refers to an aryl group that has been substituted with an alkyl group as defined above, with 4-methylphenyl being an exemplary alkylaryl group; “arylalkyl” refers to an alkyl group that has been substituted with an aryl group as defined above, with benzyl being an exemplary arylalkyl group; “acyl” refers to an alkyl group as defined above with the indicated number of carbon atoms attached through a carbonyl carbon bridge (—C(═O)—); “alkoxy” refers to an alkyl group as defined above with the indicated number of carbon atoms attached through an oxygen bridge (—O—); and “aryloxy” refers to an aryl group as defined above with the indicated number of carbon atoms attached through an oxygen bridge (—O—).

Unless otherwise indicated, each of the foregoing groups can be unsubstituted or substituted, provided that the substitution does not significantly adversely affect synthesis, stability, or use of the compound. The term “substituted” as used herein means that at least one hydrogen on the designated atom or group is replaced with another group, provided that the designated atom's normal valence is not exceeded. When the substituent is oxo (i.e., =0), then two hydrogens on the atom are replaced. Combinations of substituents and/or variables are permissible provided that the substitutions do not significantly adversely affect synthesis or use of the compound. Exemplary groups that can be present on a “substituted” position include, but are not limited to, cyano; hydroxyl; nitro; azido; alkanoyl (such as a C₂₋₆ alkanoyl group such as acyl); carboxamido; C₁₋₆ or C₁₋₃ alkyl, cycloalkyl, alkenyl, and alkynyl (including groups having at least one unsaturated linkages and from 2 to 8, or 2 to 6 carbon atoms); C₁₋₆ or C₁₋₃ alkoxys; C₆₋₁₀ aryloxy such as phenoxy; C₁₋₆ alkylthio; C₁₋₆ or C₁₋₃ alkylsulfinyl; C1-6 or C₁₋₃ alkylsulfonyl; aminodi(C₁₋₆ or C₁₋₃)alkyl; C₆₋₁₂ aryl having at least one aromatic rings (e.g., phenyl, biphenyl, naphthyl, or the like, each ring either substituted or unsubstituted aromatic); C₇₋₁₉ arylalkyl having 1 to 3 separate or fused rings and from 6 to 18 ring carbon atoms; or arylalkoxy having 1 to 3 separate or fused rings and from 6 to 18 ring carbon atoms, with benzyloxy being an exemplary arylalkoxy.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method of producing an aromatic chemical, comprising:
providing a feedstock comprising biomass to a first reactor to produce a first product stream, wherein the first product stream comprises methane and carbon dioxide;
combining the first product stream with a recycle stream to form a second reactor feed stream;
passing the second reactor feed stream through a second reactor to produce a second product stream comprising aromatics and hydrogen gas;
recovering aromatics from the second product stream to create a recovery stream depleted of aromatics;
combining the recovery stream with a stream comprising carbon dioxide to form a combined recovery stream;
passing the combined recovery stream to a third reactor to produce the recycle stream comprising gas; and forming an aromatic chemical from the second product stream.

2. The method of claim 2, wherein the biomass comprises a material selected from vegetation, an aquatic crop, forestry, agricultural residue, animal waste, or a combination comprising at least one of the foregoing.

3. The method of claim 1, wherein the aromatic chemical is benzene, toluene, xylene, naphthalene, or a combination comprising at least one of the foregoing.

4. The method of claim 1, wherein the gas comprises methane.

5. The method of claim 1, wherein the recycle stream further comprises water; and further comprising separating the water from the recycle stream.

6. The method of claim 1, wherein the second reactor is a dehydroaromatization reactor.

7. The method of claim 1, wherein the first product stream comprises 55 wt. % to 70 wt. % methane and 40 wt. % to 45 wt. % carbon dioxide.

8. The method of claim 1, further comprising reacting the second reactor feed stream with a catalyst in the second reactor to form the second product stream.

9. The method of claim 8, wherein the catalyst comprises a metal catalyst.

10. The method of claim 9, wherein the metal is selected from molybdenum, tungsten, ruthenium, iron, cobalt, nickel, copper, silver, zinc, chromium, tin, or a combination comprising at least one of the foregoing.

11. The method of claim 1, further comprising dehydrogenating the second product stream and/or cyclizating of the second product stream.

12. The method of claim 13, wherein the dehydrogenation and/or cyclization of the second product occurs at a temperature of 400° C. to 1,000° C. and a pressure of 0.02 MegaPascals to 0.5 MegaPascals.

13. The method of claim 12, wherein the dehydrogenation and/or cyclization of the second product occurs at a pressure or gaseous hourly space velocity of the feed gas measured in volumes of gas per volume of catalyst per hour of 400 gaseous hourly space velocity to 8,000 gaseous hourly space velocity.

14. The method of claim 1, further comprising contacting a methanation catalyst with the combined recovery stream to produce the third product stream.

15. The method of claim 14, wherein the methanation catalyst is selected from ruthenium, cobalt, nickel, iron, or a combination comprising at least one of the foregoing.

16. The method of claim 1, wherein the third product stream is formed at a temperature of 200° C. to 600° C. and pressure of 0 MegaPascals to 75 MegaPascals.

17. A method of producing an aromatic chemical, comprising:
providing a feedstock comprising biomass to a first reactor to produce a first product stream, wherein the first product stream comprises methane and carbon dioxide;
combining the first product stream with a recycle stream to form a second reactor feed stream;
passing the second reactor feed stream through a dehydroaromatization reactor to produce a second product stream comprising aromatics and hydrogen gas;
recovering aromatics from the second product stream to create a recovery stream depleted of aromatics;
combining the recovery stream with a stream comprising carbon dioxide to form a combined recovery stream;
passing the combined recovery stream to a third reactor to produce a third product stream comprising water and gas;
forming an aromatic chemical from the second product stream; and
recovering methane from the third product stream to form the recycle stream.

18. A method of producing an aromatic chemical, comprising:
supplying a feedstock comprising biomass to a digester, wherein digestion occurs at 20° C. to 50° C. to form a first product stream;
passing the first reactor outlet stream to a first separator, wherein the first reactor outlet stream comprises 55 wt. % to 70 wt. % methane and 30 wt. % to 45 wt. % carbon dioxide and wherein the first separator separates the first reactor outlet stream into a first product stream comprising methane and a diverted stream comprising carbon dioxide;
recovering the first product stream from the first separator;
combining the first product stream with a recycle stream to form a second reactor feed stream;
passing the second reactor feed stream through a second reactor to convert the methane to aromatics and hydrogen through a dehydrocyclization reaction and to hydrocarbons with a dehydrogenation-coupling reaction in the second reactor to form a second product stream;
feeding the second product stream to a condenser to separate the aromatics from the second product stream to form an aromatic stream and an aromatic depleted product stream;
combining the aromatic depleted product stream with hydrogen to form a combined recovery stream;
sending the combined recovery stream to a methanation reactor to form a third product stream;
feeding the third product stream to a second separator; and
separating the third product stream to form a stream comprising water and the recycle stream comprising methane in the second separator.

19. The method of claim 18, further comprising contacting the second reactor feed stream with a catalyst in the second reactor.

20. The method of claim 18, further comprising adding the reaction products of the first product stream to the methanation reactor to produce the third product stream.
